(19) United States
(12) Patent Application Publication
(10) Pub. No.: US 2023/0238199 A1
(43) Pub. Date: Jul. 27, 2023

(54) APPARATUS FOR STERILIZING ELECTRICAL SWITCHES AND ELECTRICAL SWITCH COVER PLATES WITH SANITIZING ULTRA VIOLET C STERILIZING GERMICIDAL LIGHT

(71) Applicant: Tom Chi, Wichita, KS (US)

(72) Inventor: Tom Chi, Wichita, KS (US)

(21) Appl. No.: 18/010,543

(22) PCT Filed: Jun. 17, 2021
(86) PCT No.: PCT/US2021/037871

§ 371 Date: Dec. 15, 2022

Related U.S. Application Data
(60) Provisional application No. 63/040,663, filed on Jun. 18, 2020.

(51) Int. Cl. H01H 23/04; A61L 2/10

(52) U.S. Cl. CPC H01H 23/04; A61L 2/10

(57) ABSTRACT

An apparatus for sanitizing electric rocker switches includes an electric rocker switch with sanitizing ultraviolet C sterilizing germicidal light which provides a germ-free surface. Sanitizing ultraviolet C sterilizing germicidal light electric emitters are incorporated within the electrical rocker switch and/or around the perimeter of the electrical rocker switch and/or electrical switch plate that surrounds the electrical rocker switch.

### BACKGROUND OF THE INVENTION

### Technical Field of the Invention

The present invention relates to an apparatus for sterilizing electrical switches and electrical switch cover plates with sanitizing ultraviolet C sterilizing germicidal light by the introduction of sanitizing ultraviolet C sterilizing germicidal light to the surfaces of electrical switches and switch plates to sanitize the touching surfaces for a germ-free environment.

### Description of the Prior Art

It is well established that germs from hands are easily communicated to a person's face especially when the hands touch the face several times a minute, as for example the eyes are rubbed, the nose is touched and other times when the hands consciously and unconsciously touch the face. Since the exposed face, more than any other part of the human body, is more susceptible to transmitting germs to the body's systems it imperative to maintain as best as possible a germ free face and hands free of pathogens which can enter the eyes, ears, mouth, nose etc.

The prior art known to the inventor is represented by the following: Littles, U.S. Pat. No. 4,777,570, issued Oct. 11, 1988; Geringer et al., U.S. Patent Application Publication No. 2016/0273243, published Sep. 22, 2016; Martin et al., U.S. Patent Application Publication No. 2018/0080253, published Mar. 22, 2018; Martin et al., U.S. Patent Application Publication No. 2018/0171669, published Jun. 21, 2018; and, Gerhardt et al., U.S. Patent Application Publication No. 2019/0342443.

### SUMMARY OF THE INVENTION

A primary object of the present invention is to incorporate into electrical switches and switch plate covers sanitizing ultraviolet C sterilizing germicidal light means for sterilization purposes.

Another object of the present invention is to utilize the electric power of the switch itself to power the provide the electrical power to energize the source of ultraviolet C sterilizing germicidal light.

The source of the ultraviolet C sterilizing germicidal light may be from, for example, one or more light emitting diodes, fiber optics, glass fibers, mercury bulbs or any other source and with the invention preferably being implemented into such electrical rocker switched, electrical toggle switched and alike.

A still further object of the invention is to provide means so that the system is automatically energized.

The foregoing and related objects are accomplished by the present invention, which provides an apparatus for sanitizing electrical switches with sanitizing ultraviolet C sterilizing germicidal light to provide a germ-free touching surface, which apparatus includes an electrical switch having a housing touching area whereby a user physically comes into physical contact, an electrical wall plate associated with the electrical switch, and one or more electric emitters that sanitizing ultraviolet C sterilizing germicidal light onto the touching area of the electrical switch.

Preferably, the touching area of the electrical rocker switch is translucent so that the electric emitters that emit sanitizing ultraviolet C sterilizing germicidal light illuminate the touching surface to provide a germ-free area.

Preferably, the electric emitters that emit sanitizing ultraviolet C sterilizing germicidal light are located around the perimeter of the electrical rocker switch and/or around the electrical switch plate.

It is also preferable that the electric emitters that emit sanitizing ultraviolet C sterilizing germicidal light are internally electrically powered by the same electrical power that powers the electrical rocker switch.

By providing sanitizing ultraviolet C sanitizing germicidal light to electrical switches and electrical switch cover plates results in a germ-free surface by disinfecting and sterilizing electrical switches and electrical switch plates that are touched by a user hands. The hygienic reason for exposing electrical switches and electrical switch plates to ultraviolet C sterilizing germicidal light sterilizing light is to kill or inactivate microorganisms by destroying nucleic acids, disrupting their DNA/RNA and leaving them unable to function or reproduce.

The source of the ultraviolet C sterilizing germicidal light may be from one or more light emitting diodes, fiber optics, glass fibers, mercury bulbs or any other source.

The invention maybe implemented into such electrical rocker switched, electrical toggle switched and alike.

Other objects and features of the present invention will become apparent when considered in combination with the accompanying drawing figures, which illustrate certain preferred embodiment of the present invention. It should, however, be noted that the accompanying drawing figures are intended to illustrate only select preferred embodiments of the claimed invention and are not intended as a means for defining the limits and scope of the invention.

### BRIEF DESCRIPTION OF THE DRAWING FIGURES

In the drawing figures, wherein similar features are denoted with similar reference numerals throughout the several views:

FIG. 1 is a front elevational view of the apparatus for sterilizing electrical switches and electrical switch cover plates with sanitizing ultraviolet C sterilizing germicidal light that has incorporated within the confines of the electrical switch sanitizing ultraviolet C sterilizing germicidal light;

FIG. 2 is front elevational view of the apparatus for sterilizing electrical switches and electrical switch cover plates with sanitizing ultraviolet C sterilizing germicidal light that has incorporated within the confines of the electrical switch and electrical switch cover plate sanitizing ultraviolet C sterilizing germicidal light;

FIG. 3 is another front elevational view of the apparatus for sterilizing electrical switches and electrical switch cover plates with sanitizing ultraviolet C sterilizing germicidal light that has incorporated within the confines of the electrical switch and electrical switch cover plate sanitizing ultraviolet C sterilizing germicidal light;

FIG. 4 is a summary of the apparatus for sterilizing electrical switches and electrical switch cover plates with sanitizing ultraviolet C sterilizing germicidal light that has incorporated within the confines of the electrical switch and electrical switch cover plate sanitizing ultraviolet C sterilizing germicidal light;

FIG. 5 is a front elevational view of the apparatus for sterilizing electrical switches and electrical switch cover plates with sanitizing ultraviolet C sterilizing germicidal light illustrating a rocker switch;

FIG. 6 is a front elevational view of the apparatus for sterilizing electrical switches and electrical switch cover plates with sanitizing ultraviolet C sterilizing germicidal light illustrating a toggle switch;

FIG. 7 is a front elevational view of the apparatus for sterilizing electrical switches and electrical switch cover plates with sanitizing ultraviolet C sterilizing germicidal light illustrating an electrical rocker switch showing the coverage of the sanitation ultraviolet C sterilizing germicidal light;

FIG. 8 is an exploded front elevational view of the apparatus for sterilizing electrical switches and electrical switch cover plates with sanitizing ultraviolet C sterilizing germicidal light illustrating an electrical rocker switch;

FIG. 9 is an exploded side elevational view of the apparatus for sterilizing electrical switches and electrical switch cover plates with sanitizing ultraviolet C sterilizing germicidal light illustrating an electrical rocker switch;

FIG. 10 is an exploded side elevational view of the apparatus for sterilizing electrical switches and electrical switch cover plates with sanitizing ultraviolet C sterilizing germicidal light illustrating an electrical rocker switch;

FIG. 11 is a perspective view of the apparatus for sterilizing electrical switches and electrical switch cover plates with sanitizing ultraviolet C sterilizing germicidal light illustrating an electrical rocker switch;

FIG. 12 is a perspective view of the apparatus for sterilizing electrical switches and electrical switch cover plates with sanitizing ultraviolet C sterilizing germicidal light illustrating an electrical rocker switch;

FIG. 13 is a front elevation and side elevation view of the apparatus for sterilizing electrical switches and electrical switch cover plates with sanitizing ultraviolet C sterilizing germicidal light illustrating an electrical rocker switch;

FIG. 14 is a front elevational perspective view of the apparatus for sterilizing electrical switches and electrical switch cover plates with sanitizing ultraviolet C sterilizing germicidal light illustrating an electrical rocker switch;

FIG. 15 is a front elevational view of the apparatus for sterilizing electrical switches and electrical switch cover plates with sanitizing ultraviolet C sterilizing germicidal light illustrating an electrical rocker switch; and,

FIG. 16 is a front elevational view of the apparatus for sterilizing electrical switches and electrical switch cover plates with sanitizing ultraviolet C sterilizing germicidal light illustrating the cover plate for an electrical rocker switch with the electrical rocker switch removed.

### DESCRIPTION OF THE REFERENCE NUMERALS

10 wall

12 electrical switch plate

14 electrical rocker switch

16 sanitizing ultraviolet C sterilizing germicidal light electric emitter embedded into touching surface of electrical rocker switch

16a sanitizing ultraviolet C sterilizing germicidal light electric emitter located around the perimeter of electrical rocker switch

16b sanitizing ultraviolet C sterilizing germicidal light electric emitter located around the perimeter of the electrical switch plate

18 sanitizing ultraviolet C sterilizing germicidal light

20 translucent germ free touching area of electrical rocker switch

22 electrical toggle switch

24 external power source

### DETAILED DESCRIPTION OF THE DRAWING FIGURES AND PREFERRED EMBODIMENTS

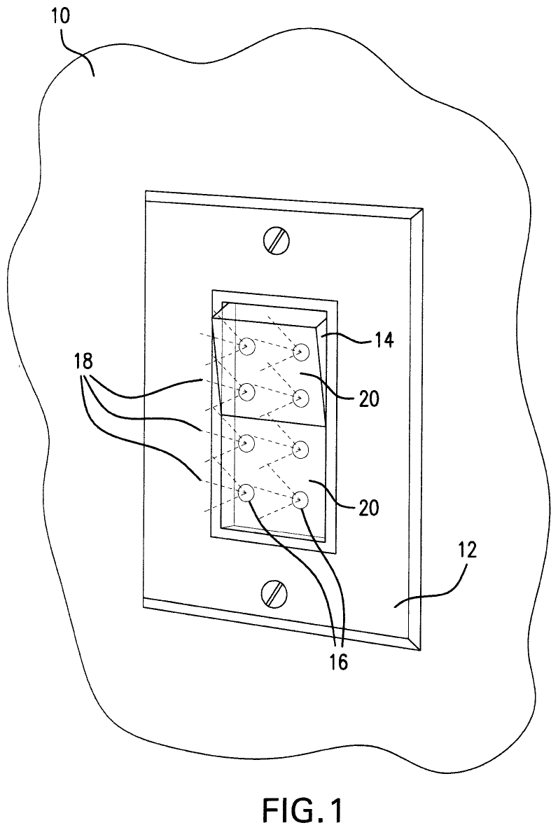

Turning now, in detail to the accompanying drawing figures, FIG. 1 shows the structure of the sanitizing ultraviolet C sterilizing germicidal light of the present invention wherein electric rocker switch 14 is integrated with sanitizing ultraviolet C sterilizing germicidal light emitters 16. The integrated sanitizing ultraviolet C sterilizing germicidal light emitters 16 of electrical rocker switch 14 are powered by electric current from the same electric power source that connects to electric rocker switch 14 and/or from external power source 24.

The touching area of electrical rocker switch 14 is constructed from translucent material so that the sanitizing ultraviolet C sterilizing germicidal light emitted from sanitizing ultraviolet C sterilizing germicidal light emitter 16 contacts the user's hand and accordingly provides germ-free touching area 20 of electrical rocker switch 14. Sanitizing ultraviolet C sterilizing germicidal light emitters 16 are spaced within electrical rocker switch 14 so that the touching area 20 of electrical rocker switch 14 is covered with sanitizing ultraviolet C sterilizing germicidal light 18 for a germ-free surface 20.

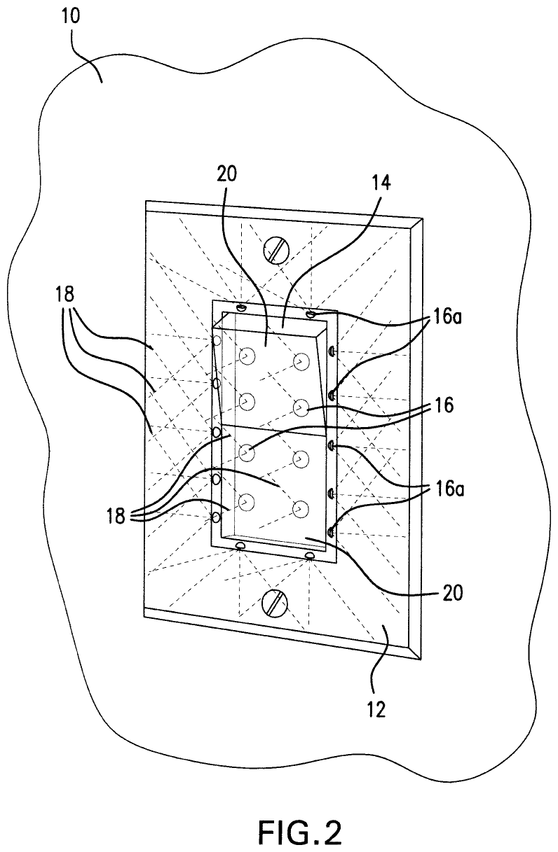

FIG. 2 is a front elevational view of electrical switch plate 12 and positioned around the periphery of electrical rocker switch 14 are sanitizing ultraviolet C sterilizing germicidal light emitters 16a that emit sanitizing ultraviolet C sterilizing germicidal light that covers the touching area 20 of electrical rocker switch 14.

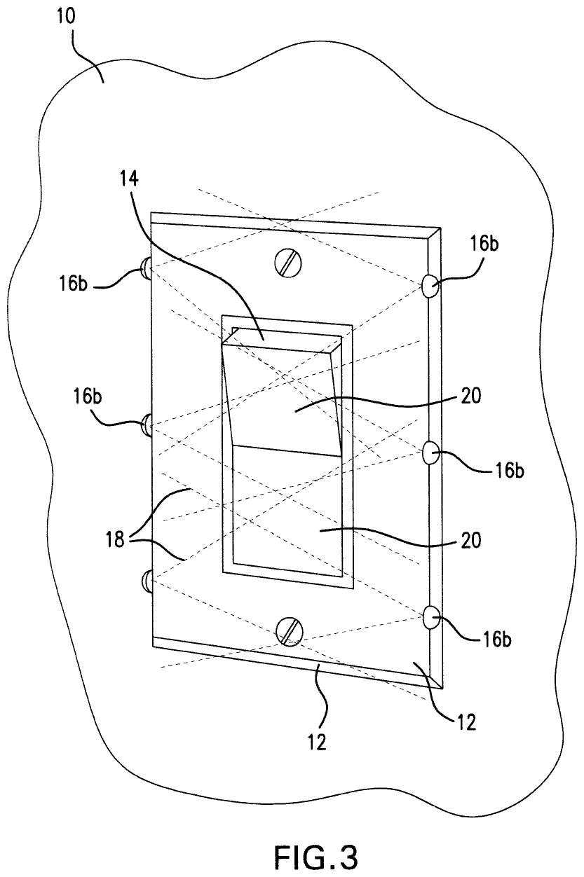

FIG. 3 is a front elevational view of electrical switch plate 12 and positioned around the periphery of electrical switch plate 12 are sanitizing ultraviolet C sterilizing germicidal light emitters 16b that emit sanitizing ultraviolet C sterilizing germicidal light that covers the touching area 20 of electrical rocker switch 14.

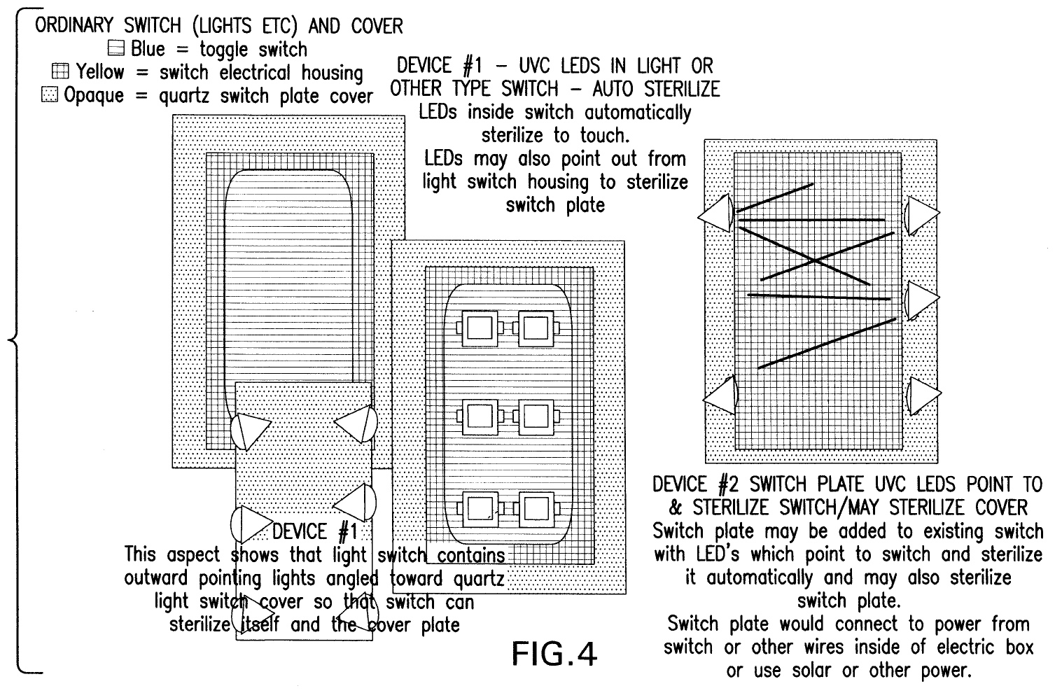

FIG. 4 shows the general structure of the sanitizing ultraviolet C sterilizing germicidal light of the present invention wherein electric rocker switch 14 is integrated with sanitizing ultraviolet C sterilizing germicidal light emitters.

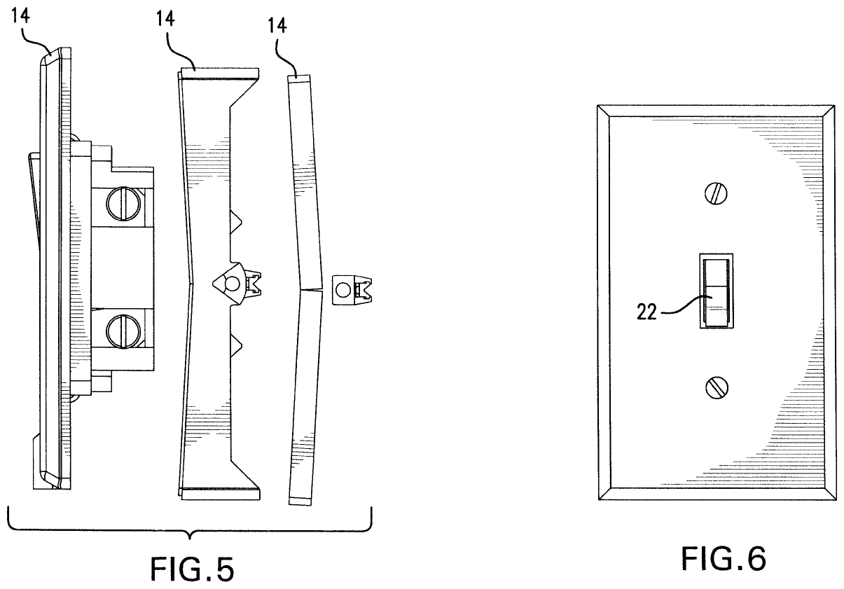

FIG. 5 is a side elevational exploded view of a typical electrical rocker switch 14 indicating various components.

FIG. 6 is a front elevational view of a typical electrical toggle switch 22.

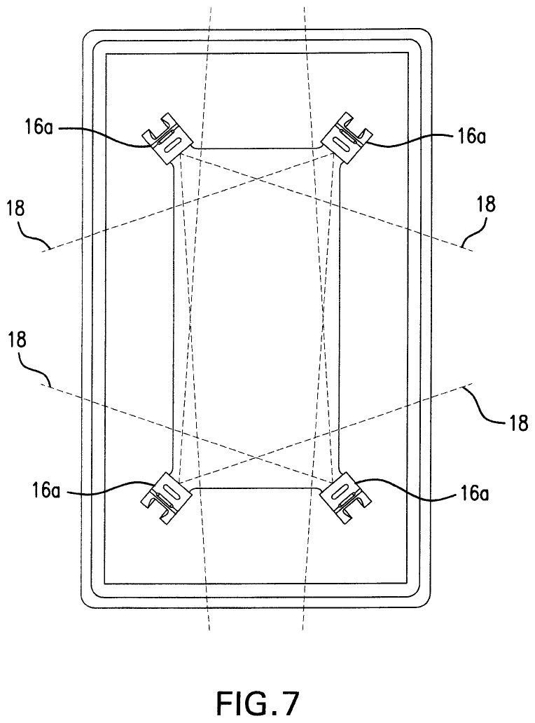

FIG. 7 is a front elevational view showing the structure of the sanitizing ultraviolet C sterilizing germicidal light of the present invention wherein electric rocker switch 14 is sanitized and disinfected with sanitizing ultraviolet C sterilizing germicidal light by emitters 16a.

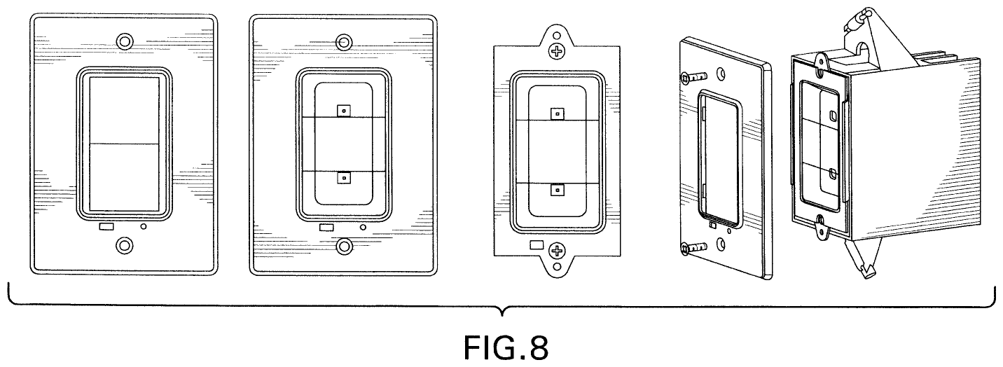

FIG. 8 is a front elevational plan and exploded view of a typical electrical rocker switch 14 indicating various components.

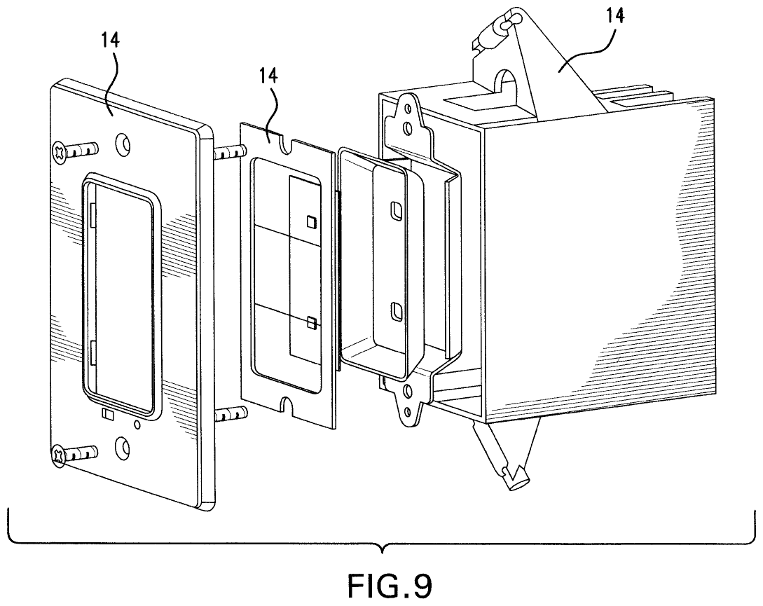

FIG. 9 is a front elevational exploded plan view of a standard electrical rocker switch 14 indicating various components.

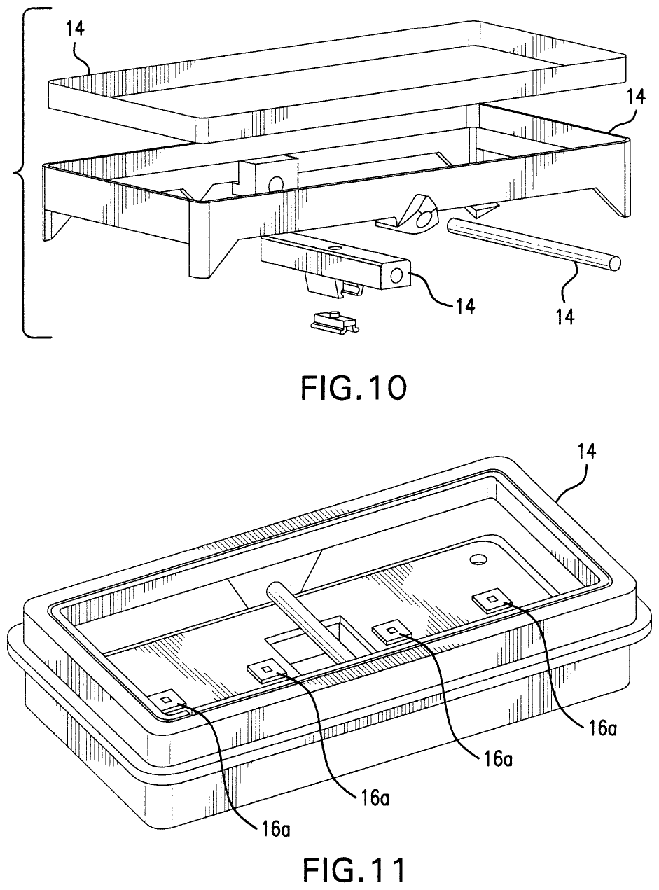

FIG. 10 is a right side elevational exploded plan view of a standard electrical rocker switch 14 indicating various components.

FIG. 11 is a top plan perspective view of a typical electrical rocker switch 14 indicating various components with sanitizing ultraviolet C sterilizing germicidal light emitters 16a located around the perimeter of electrical rocker switch 14.

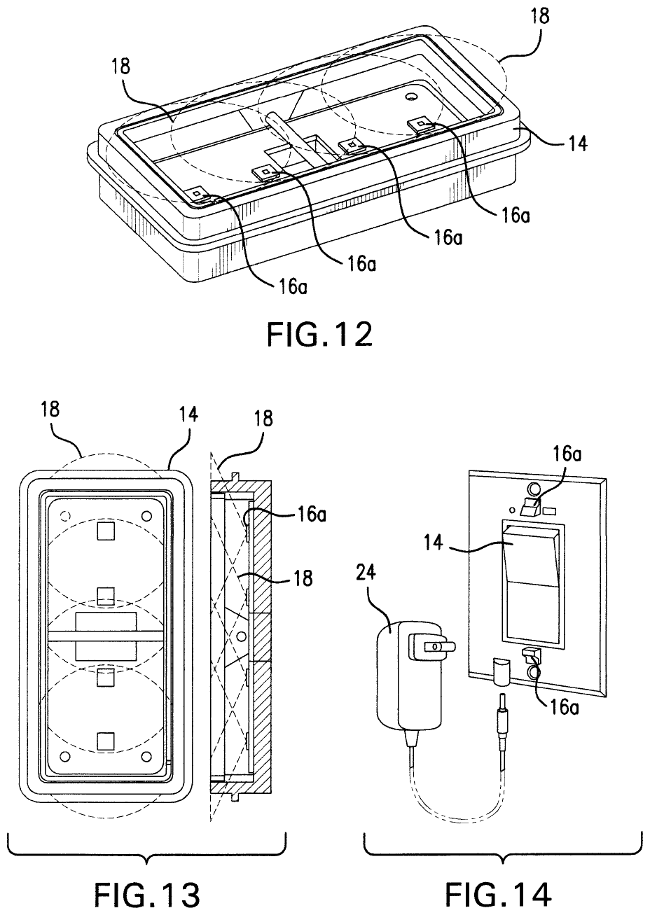

FIG. 12 is a top plan perspective view of a typical electrical rocker switch 14 indicating various components with sanitizing ultraviolet C sterilizing germicidal light emitters 16a located around the perimeter of electrical rocker switch 14 and sanitizing ultraviolet C sterilizing germicidal light 18 emitting from emitters 16a.

FIG. 13 is front and side view of a standard electrical rocker switch 14 indicating various components with sanitizing ultraviolet C sterilizing germicidal light emitters 16a located around the perimeter of electrical rocker switch 14 and sanitizing ultraviolet C sterilizing germicidal light 18 emitting from emitters 16a.

FIG. 14 is front perspective elevational view of a typical electrical rocker switch 14 indicating various components with sanitizing ultraviolet C sterilizing germicidal light emitters 16a located around the perimeter of electrical rocker switch 14 and power source 24 connected to electrical rocker switch 14.

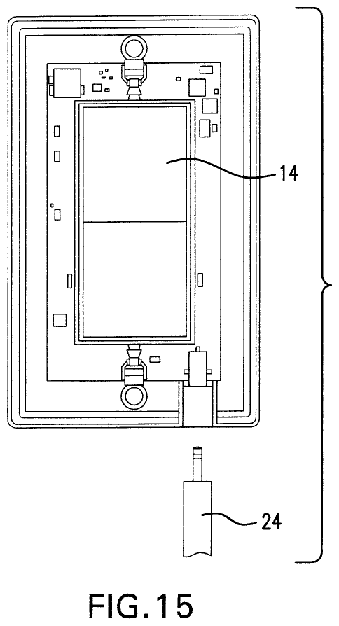

FIG. 15 is front elevational view of a typical electrical rocker switch 14 indicating various and power source 24 connected to electrical rocker switch 14.

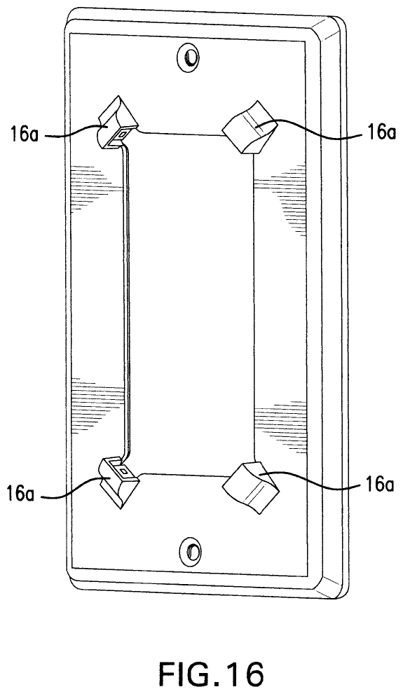

FIG. 16 is front perspective elevational view of a typical electrical rocker switch 14 indicating various components with sanitizing ultraviolet C sterilizing germicidal light emitters 16a located around the perimeter of electrical rocker switch 14.

While only several embodiments of the present invention have been shown and described, it will be obvious to those skilled in the art that many modifications may be made to the present invention without departing from the spirit and scope thereof.

1. An apparatus for sanitizing electrical switches with sanitizing ultraviolet C sterilizing germicidal light to provide a germ-free touching surface, comprising:
an electrical switch having a housing touching area wherein a user is able to physically comes into contact with said housing touching area;
an electrical wall plate associated for said electrical switch; and,
an electric emitter that emits sanitizing ultraviolet C sterilizing germicidal light onto said touching area of said electrical switch, wherein said touching area of said electrical switch is translucent so that said electric emitter that emits sanitizing ultraviolet C sterilizing germicidal light illuminates said touching surface for providing a germ-free area.

2. (canceled)

3. The apparatus for sanitizing electrical switches with sanitizing ultraviolet C sterilizing germicidal light to provide a germ-free touching surface according to claim 1, wherein the electric emitter that emits sanitizing ultraviolet C sterilizing germicidal light is located around a perimeter of the electrical switch.

4. The apparatus for sanitizing electrical switches with sanitizing ultraviolet C sterilizing germicidal light to provide a germ-free touching surface according to claim 1, wherein the electric emitter that emits sanitizing ultraviolet C sterilizing germicidal light is located around a perimeter of the electrical switch plate.

5. The apparatus for sanitizing electrical switches with sanitizing ultraviolet C sterilizing germicidal light to provide a germ-free touching surface according to claim 1, wherein the electric emitter that emits sanitizing ultraviolet C sterilizing germicidal light is internally electrically powered by the same electrical power that powers said electrical switch.

6. The apparatus for sanitizing electrical switches with sanitizing ultraviolet C sterilizing germicidal light to provide a germ-free touching surface according to claim 1, wherein the electric emitter that emits sanitizing ultraviolet C sterilizing germicidal light is powered by an external power source.

7. The apparatus for sanitizing electrical switches with sanitizing ultraviolet C sterilizing germicidal light to provide a germ-free touching surface according to claim 1, wherein said electrical switch is an electrical rocker switch.
